(19) United States
(12) Patent Application Publication
(10) Pub. No.: US 2024/0180435 A1
(43) Pub. Date: Jun. 6, 2024

(54) BLOOD PRESSURE MEASUREMENT DEVICE

(71) Applicant: OMRON HEALTHCARE Co., Ltd., Kyoto (JP)

(72) Inventors: Yasuhiro KAWABATA, Kyoto (JP); Hiroyuki KANDA, Kyoto (JP); Tatsunori ITO, Kyoto (JP); Yuta KUDO, Kyoto (JP)

(21) Appl. No.: 18/442,585

(22) Filed: Feb. 15, 2024

Related U.S. Application Data
(63) Continuation of application No. PCT/JP2022/044819, filed on Dec. 6, 2022.

(30) Foreign Application Priority Data
Dec. 9, 2021 (JP) 2021-199914

(51) Int. Cl. A61B 5/021; A61B 5/00; A61B 5/02

(52) U.S. Cl. CPC A61B 5/02116; A61B 5/02028; A61B 5/7282; A61B 5/742; A61B 5/7455; A61B 5/746

(57) ABSTRACT

A blood pressure measurement device includes: a pulse wave acquisition unit configured to acquire a pulse wave of a human body; a blood pressure value measurement unit configured to calculate a blood pressure value based on the pulse wave; a pulse wave reproduction information generation unit configured to generate pulse wave reproduction information, the pulse wave reproduction information being dynamic information reproducing pulsation of the pulse wave at a time of calculation of the blood pressure value; a storage unit configured to store the pulse wave reproduction information; a display unit configured to be capable of displaying the blood pressure value calculated; and an output unit configured to be capable of outputting the pulse wave reproduction information. The output unit can output the pulse wave reproduction information related to the pulse wave at the time of calculation of the blood pressure value when the blood pressure value is displayed.

### CROSS-REFERENCE TO RELATED APPLICATIONS

This application is the U.S. national stage application filed pursuant to 35 U.S.C. 365(c) and 120 as a continuation of International Patent Application No. PCT/JP2022/044819, filed Dec. 6, 2022, which application claims priority to Japanese Patent Application No. 2021-199914, filed Dec. 9, 2021, which applications are incorporated herein by reference in their entireties.

### TECHNICAL FIELD

The present invention belongs to the technical field related to healthcare, and particularly relates to a blood pressure measurement device.

### BACKGROUND ART

In recent years, it has become widespread to perform health management by measuring information related to the body and health of an individual such as a blood pressure value with a measurement device and recording and analyzing the measurement result. As an example of the above-described measurement device, a blood pressure monitor which is widely used in general households for measurement in daily life for a long period of time is well-known.

In this respect, blood pressure monitors having a variety of functions have been suggested, and, for example, a blood pressure monitor including a display unit capable of displaying a signal level of a pulse wave detected from the start to the end of measurement of a blood pressure value in a time-series graph is publicly known (Patent Document 1).

According to the blood pressure monitor described in Patent Document 1, since a time-series graph of a signal level of a pulse wave is displayed as a time-series pulse wave graph, it is possible to clearly indicate what pulse wave is detected by a blood pressure calculation unit and what feature point of the detected pulse wave is used by the blood pressure calculation unit to calculate a blood pressure value. Therefore, it is possible to visually confirm that no noise is superimposed on the pulse wave, and to confirm whether the blood pressure value is normally calculated.

### CITATION LIST

### Patent Literature

Patent Document 1: JP 2007-98003 A

### SUMMARY OF INVENTION

### Technical Problem

Incidentally, an arrhythmia such as atrial fibrillation (AF) that indicates a sign of a serious disease such as cerebral infarction can be detected based on information related to a pulse wave (in particular, pulse wave intervals). As in the blood pressure monitor described in Patent Document 1, acquiring a pulse wave during measurement of a blood pressure value is a known technique, and when information about the pulse wave can be presented to a user together with the blood pressure value, the user can also check for an arrhythmia only by blood pressure measurement.

However, even when a time-series graph of a signal level of a pulse wave is displayed as in the technique described in Patent Document 1, it is difficult for a general user who does not have medical knowledge to correctly read information of an arrhythmia from the time-series graph. In addition, even when the user can read the information of the arrhythmia from the time-series graph, the user cannot sensuously recognize an arrhythmia (i.e., pulse irregularity), but only recognizes the information as objective (unrealistic) information, and may not have a sense of urgency.

The present invention has been made in view of the above circumstances, and an object of the present invention is to provide a technique that can enable a user who has no medical knowledge to sensuously recognize an arrhythmia and enhance the user's recognition about an arrhythmia with a blood pressure measurement device capable of displaying information of a pulse wave.

### Solution to Problem

The present invention adopts the following configurations to solve the above-described problems. That is, a blood pressure measurement device including:
a pulse wave acquisition unit configured to acquire a pulse wave of a human body;
a blood pressure value measurement unit configured to calculate a blood pressure value based on the pulse wave;
a pulse wave reproduction information generation unit configured to generate pulse wave reproduction information, the pulse wave reproduction information being dynamic information reproducing pulsation of the pulse wave at a time of calculation of the blood pressure value;
a storage unit configured to store the pulse wave reproduction information;
a display unit configured to display the blood pressure value calculated; and
an output unit configured to output the pulse wave reproduction information, wherein the output unit is configured to output the pulse wave reproduction information related to the pulse wave at the time of calculation of the blood pressure value when the blood pressure value is displayed at the display unit.

Note that the term “pulse wave” mentioned here is waveform information related to changes in pulsation of a blood vessel associated with blood ejection by a heart, and includes both a pressure pulse wave and a volume pulse wave. The blood pressure value measurement unit may be any unit that calculates a blood pressure value using a pulse wave, and a calculation method thereof may be an oscillometric method or a Korotkoff method. In addition, the term “dynamic” mentioned here means being associated with changes over time.

According to the blood pressure measurement device having such a configuration, when a user checks a measured blood pressure value, the user can recognize the pulsation of a pulse wave at the time of measurement of the blood pressure value as dynamic information. Therefore, when there is an arrhythmia, the arrhythmia can be recognized intuitively and sensuously.

The storage unit may store the blood pressure value calculated, the display unit may be configured to display the blood pressure value stored in the storage unit, and the output unit may output the pulse wave reproduction information at the time of calculation of the blood pressure value when the blood pressure value stored is displayed at the display unit.

According to this, information reproducing pulsation at the time of blood pressure measurement in the past can be recognized not only at the time of the blood pressure measurement, but also at the time of reading and checking of a result of the blood pressure measurement in the past afterward. Thus, a user can sensuously recognize the transition of pulse waves. In addition, since information of pulse waves at past blood pressure measurements can be presented at the time of a medical examination by a doctor or the like, the doctor can determine a treatment strategy based on the information of blood pressure values and pulse waves in the daily life of the user.

The blood pressure measurement device may further include an arrhythmia detection unit configured to detect an arrhythmia when the arrhythmia is present in the pulse wave, and the output unit may, only when the blood pressure value calculated based on the pulse wave at a time of detection of the arrhythmia is displayed at the display unit, output dynamic information reproducing pulsation of the pulse wave at a time of calculation of the blood pressure value.

According to this, it is possible to save the user from taking time to check information reproducing pulsation of a normal pulse wave because information reproducing pulsation of a pulse wave is output only at the time of displaying a blood pressure value with a presence of an arrhythmia. When a blood pressure value is stored in the storage unit, information of an arrhythmia detected at the time of acquisition of the blood pressure value may be stored in association with the blood pressure value.

The display unit and/or the output unit may output information indicating presence of the arrhythmia when the blood pressure value calculated based on the pulse wave at the time of detection of the arrhythmia is displayed at the display unit. According to this configuration, the user can easily recognize that the user is referring to pulse wave information (or a blood pressure value) in which an arrhythmia is present.

The pulse wave reproduction information may be dynamic information reproducing pulse wave intervals of the pulse wave. From the viewpoint of recognizing an arrhythmia, it is preferable to output such information. However, information reproduced by the output unit is not necessarily limited to only pulse wave intervals, and information related to (the magnitude of) the amplitude of a pulse may be reproduced, for example.

The display unit may also serve as the output unit, and the pulse wave reproduction information may be output as a moving image. With such a configuration, there is no need to provide a separate output unit for outputting dynamic information reproducing pulsation of a pulse wave, and the configuration of the device can be simplified.

However, the output of dynamic information reproducing pulsation of a pulse wave is not limited to an output based on a moving image. For example, pulsation of a pulse wave can be reproduced by generating sound or vibration, or pulsation of a pulse wave may be reproduced by combining a plurality of outputs of image, sound, and vibration.

Also, the configurations and processing described above can be combined with one another to constitute the present invention unless the combination leads to contradiction.

### Advantageous Effects of Invention

According to the present invention, it is possible to provide a technique that can enable a user who has no medical knowledge to sensuously recognize an arrhythmia and enhance the user's recognition about an arrhythmia with a blood pressure measurement device capable of displaying information of a pulse wave.

### BRIEF DESCRIPTION OF DRAWINGS

FIG. 1 is a schematic diagram illustrating an outline of a device configuration and a functional configuration of a blood pressure measurement device according to an embodiment.

FIG. 2A is a first diagram illustrating an example of a screen displayed at an image display portion of the blood pressure measurement device according to the embodiment. FIG. 2B is a second diagram illustrating an example of a screen displayed at the image display portion of the blood pressure measurement device according to the embodiment.

FIG. 3A is a first diagram illustrating a relationship between a pulse wave detected during blood pressure measurement and blinking timing of a pulse wave icon displayed at the image display portion. FIG. 3B is a second diagram illustrating a relationship between a pulse wave detected during blood pressure measurement and blinking timing of a pulse wave icon displayed at the image display portion. FIG. 3C is a third diagram illustrating a relationship between a pulse wave detected during blood pressure measurement and blinking timing of a pulse wave icon displayed at the image display portion.

### DESCRIPTION OF EMBODIMENTS

### First Embodiment

Embodiments of the present invention will be specifically described below with reference to the drawings. It should be noted that the material, shape, relative arrangement, and the like of configurations described in this embodiment are not intended to limit the scope of this invention to the configurations alone, unless otherwise stated.

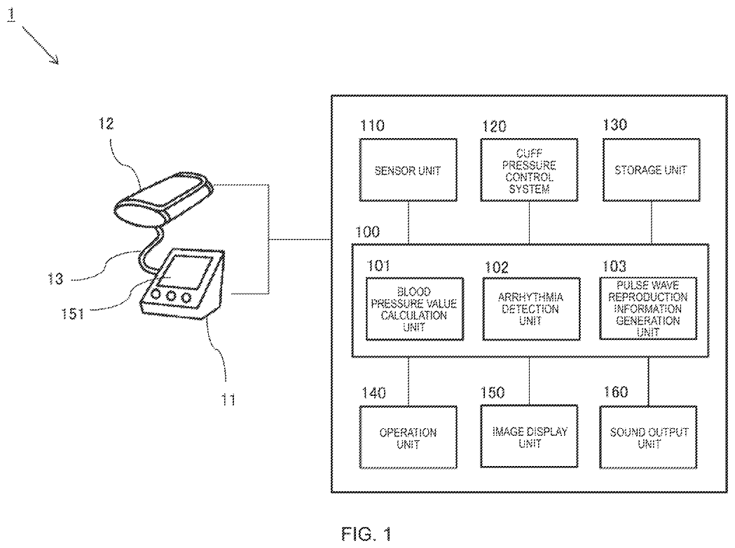

FIG. 1 is a schematic diagram illustrating an outline of the device configuration and the functional configuration of a blood pressure measurement device 1 according to the present embodiment. As illustrated in FIG. 1, the blood pressure measurement device 1 generally includes a main body portion 11, a cuff portion 12, and an air tube 13. As illustrated by the functional blocks in FIG. 1, the blood pressure measurement device 1 includes functional units of a control unit 100, a sensor unit 110, a cuff pressure control system 120, a storage unit 130, an operation unit 140, an image display unit 150, and a sound output unit 160.

Although not illustrated, the main body portion 11 includes an image display portion 151 such as a liquid crystal display (LCD), various operation buttons, a sound output portion such as a speaker, a power supply portion such as a battery, a pump and a valve communicating with the cuff portion, a housing accommodating these components, and the like. The cuff portion 12 is a member used by being wrapped around an upper arm of a user, and includes an air bag (cuff) communicating with the pump and the valve of the main body portion 11 via the air tube 13, a belt incorporating the cuff, a pressure sensor provided at the belt (none of which are illustrated), and the like. When blood pressure measurement is performed by the Korotkoff method, a microphone may be included.

The belt of the cuff portion 12 is provided with a fixing member (for example, a hook-and-loop fastener) for fixing the cuff portion 12 to an upper arm of the user, and the cuff portion 12 is wound around the upper arm of the user by the belt when blood pressure measurement is performed using the blood pressure measurement device 1.

The control unit 100 is a unit for controlling the blood pressure measurement device 1, and includes, for example, a central processing unit (CPU). Upon receiving operation by the user via the operation unit 140, the control unit 100 controls each component of the blood pressure measurement device 1 to execute various types of processing such as blood pressure measurement and provision of various types of information in accordance with a predetermined program. Note that the predetermined program is stored in the storage unit 130 described below to be read therefrom. The control unit 100 includes a blood pressure value calculation unit 101, an arrhythmia detection unit 102, and a pulse wave reproduction information generation unit 103 as functional modules. These functional modules will be described in detail below.

The sensor unit 110 includes the pressure sensor (for example, a piezoresistive sensor including a piezoelectric element) provided at the cuff portion as described above, and detects at least a pulse wave of the user. The sensor unit 110 may include a sensor other than the pressure sensor, and may include a photoplethysmography (PPG) sensor when a pulse wave is detected by a photoelectric method. That is, in the present embodiment, the sensor unit 110 corresponds to the pulse wave acquisition unit.

The cuff pressure control system 120 controls the pump and the valve of the main body portion 11 to adjust a cuff pressure of the cuff portion 12 at the time of blood pressure measurement. Specifically, at the time of blood pressure measurement, control is performed such that the pump is driven in a state where the cuff portion 12 is wrapped around the upper arm to feed air into the cuff so as to inflate the cuff (increase a cuff pressure). In this way, the blood flow is once blocked by compressing the blood vessel of the upper arm of the user, and then control is performed such that the pump is stopped and the valve is opened to gradually release air from the cuff so as to deflate the cuff (decrease the cuff pressure).

The storage unit 130 includes a main storage device such as a random access memory (RAM), a read only memory (ROM), or the like, and auxiliary storage device such as a hard disk drive (HDD), a flash memory, or the like, and stores various types of information such as application programs, various type of measurement results such as blood pressure values, pulse waves, pulse wave reproduction information described below, and other biological information acquired. Measured blood pressure values, measured pulse waves, and the like are stored in the storage unit 130 in association with time information such as acquisition times and measurement times. As the time information, for example, information measured with reference to a real time clock (RTC) can be used. In addition, the auxiliary storage device may be configured to be attachable to and detachable from the main body portion 11.

The operation unit 140 includes components such as a power button, measurement execution button, and a selection and determination button, for example, and has a function of receiving input operation from the user and causing the control unit 100 to execute processing in response to the operation.

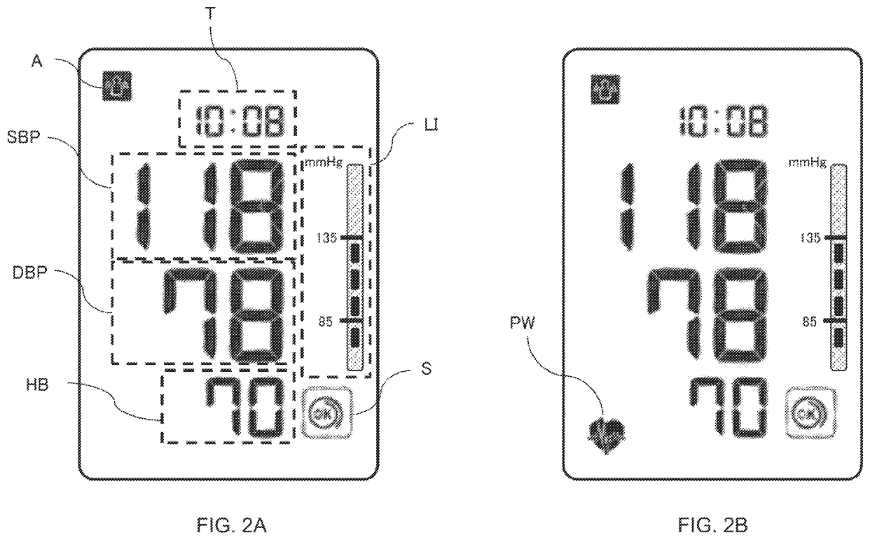

The image display unit 150 includes the image display portion 151 of the main body portion 11, and provides the user with information by displaying various types of information at the image display portion 151. FIG. 2A and FIG. 2B are examples of screens displayed at the image display portion 151 and are diagrams illustrating screens displaying blood pressure measurement results. As illustrated in FIG. 2A and FIG. 2B, at the screens displaying blood pressure measurement results, a measurement time T, a maximum (systolic) blood pressure value SBP, a minimum (diastolic) blood pressure value DBP, a pulse rate HB, a blood pressure value level indicator LI and the like are displayed. In addition, an attachment state icon S indicating whether the attachment state of the cuff portion 12 is good or bad, an arrhythmia notification icon A indicating that an arrhythmia has been detected during blood pressure measurement, a pulse wave icon PW indicating the pulsation of a pulse wave during blood pressure measurement, and the like are displayed. The arrhythmia notification icon A and the pulse wave icon PW will be further described below.

The sound output unit 160 includes a sound generation portion such as a speaker, and presents information to the user by sound. Specifically, the sound output unit 160 may generate an announcement of the start of blood pressure measurement or a guide sound related to the use of the device. As will be described below, the pulse wave reproduction information may be reproduced by sound.

Hereinafter, each functional module of the control unit 100 will be described. The blood pressure value calculation unit 101 calculates a blood pressure value (and a pulse rate) of the user based on a pulse wave acquired by the sensor unit 110. As a blood pressure calculation method, any desired known technique can be used and, for example, an oscillometric method in which a blood pressure is measured by detecting a pressure pulse wave by a pressure sensor can be employed. Alternatively, a microphone may be provided at the cuff portion 12 and the Korotkoff method for detecting Korotkoff sounds may be used. The blood pressure value and the pulse rate calculated by the blood pressure value calculation unit 101 are stored in the storage unit 130 in association with a blood pressure measurement time.

When there are disturbed pulses (arrhythmia) during blood pressure measurement, the arrhythmia detection unit 102 detects the arrhythmia, and stores information indicating the presence of the arrhythmia in the storage unit 130 in association with a measurement result at the time of the blood pressure measurement during which the arrhythmia is detected. When there is an arrhythmia, the arrhythmia notification icon A is displayed at the screen displaying the measurement result. The arrhythmia notification icon A can be displayed at both a screen displaying a measurement result immediately after measurement and a screen displaying a past measurement result.

The pulse wave reproduction information generation unit 103 acquires a pulse wave at the time of blood pressure measurement from the sensor unit 110, and stores the pulse wave in the storage unit 130 in association with time information at which the information of the pulse wave is acquired. Further, based on the information of the pulse wave, information reproducing pulsation of the pulse wave at the time of blood pressure calculation by the blood pressure value calculation unit 101 (hereinafter referred to as pulse wave reproduction information) is generated. Specifically, for example, since the pulsation can be reproduced by the blinking of the pulse wave icon PW displayed at the image display portion 151, information related to the blinking timing of the pulse wave icon PW is generated. The blinking timing of the pulse wave icon PW can be determined, for example, by acquiring peak positions of the amplitude of a pulse wave.

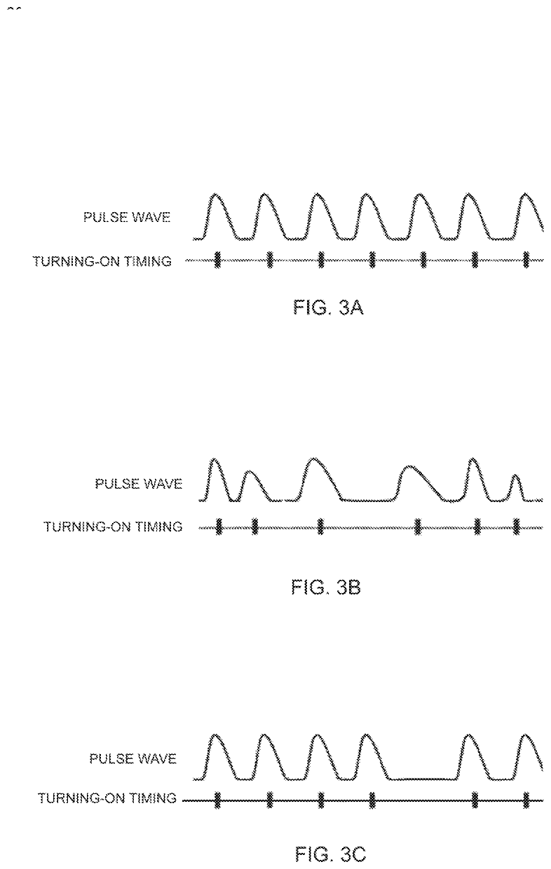

FIG. 3A, FIG. 3B, and FIG. 3C are diagrams each illustrating a relationship between a pulse wave detected during blood pressure measurement and the blinking timing of the pulse wave icon PW. FIG. 3A illustrates a normal (regular) pulse wave without arrhythmias and the blinking timing of the pulse wave icon PW reproducing the pulsation of the pulse wave. FIG. 3B illustrates a pulse wave in which an entirely irregular arrhythmias suspected of atrial fibrillation (AF) is detected, and the blinking timing of the pulse wave icon PW reproducing the pulsation of the pulse wave. FIG. 3C illustrates a pulse wave in which a (partially irregular) arrhythmia less suspected of AF is detected, and the blinking timing of the pulse wave icon PW reproducing the pulsation of the pulse wave. As can also be seen from FIG. 3A, FIG. 3B, and FIG. 3C, the blinking timing of the pulse wave icon PW in the present embodiment is synchronized with the peak positions of the amplitude of the pulse wave.

The display screen example of FIG. 2A illustrates a state in which the pulse wave icon PW is turned off, and the display screen example of FIG. 2B illustrates a state in which the pulse wave icon PW is turned on. As illustrated in FIG. 3A to FIG. 3C, the pulsation of a pulse wave can be reproduced by turning on the pulse wave icon PW (the state illustrated in FIG. 2B) at times when the amplitude of the pulse wave reaches peak positions (oscillates) and turning off the pulse wave icon PW (the state illustrated in FIG. 2A) at other times. Accordingly, when there is an arrhythmia (AF, in particular), the user can easily notice the presence of the arrhythmia by an intuitive feeling of strangeness when seeing irregular blinking of the pulse wave icon PW.

According to the blood pressure measurement device 1 having the above-described configuration, the user can check the presence or absence of disturbed pulses together with a measurement result of a blood pressure value at the time of daily blood pressure measurement, and can easily recognize a pulse irregularity when there is an arrhythmia. When there is an arrhythmia, irregular pulsation timing is reproduced in an expression that can be intuitively recognized, and thus it is possible to easily recognize not only information about the presence of an arrhythmia but also an aspect of disturbed pulses (abnormality thereof). For a measurement result whose data is kept in the storage unit 130, a blood pressure value and pulsation timing of a pulse wave at the time of measurement of the blood pressure value can be confirmed any time by reading the measurement result. The user can bring the blood pressure measurement device 1 (or only an auxiliary storage device of the storage unit 130 when the auxiliary storage device is detachable) at the time of a medical examination so as to be confirmed by a doctor. The doctor can confirm such information and determine an appropriate treatment strategy such as performing a detailed examination.

### Modified Examples

The description of the above embodiment is merely illustrative of the present invention, and the present invention is not limited to the specific embodiment described above. Within the scope of the technical idea of the present invention, various modifications and combinations may be made.

For example, in the above embodiment, when a blood pressure measurement result is displayed, the pulsation timing of a pulse wave is reproduced by blinking the pulse wave icon PW, that is, as moving image information, but the reproduction method is not limited to an image. For example, the pulsation timing of a pulse wave may be reproduced by outputting a sound (for example, an electronic sound such as “beep, beep . . . ”) from the sound output unit 160 at times when the pulse wave oscillates. This method also causes a user to have a feeling of strangeness and easily recognize an aspect of disturbed pulses because a sound is output at irregular intervals when there is an arrhythmia. The same effect can also be achieved, for example, by further providing a vibrating member for vibrating the device and generating vibration at times when a pulse wave oscillates.

Further, the reproduction of the pulsation timing of a pulse wave by sound or vibration described above may be combined with the reproduction of the pulsation timing by image described in the embodiment. This can more clearly present the pulsation timing of a pulse wave to a user.

In addition, information reproducing pulsation timing by the pulse wave icon PW is not necessarily presented every time a blood pressure measurement result is displayed, and, for example, the blinking display of the pulse wave icon PW may be performed only when a blood pressure measurement result at the time when the arrhythmia detection unit 102 detects an arrhythmia is displayed. This can prevent a situation in which time is consumed to see the regular blinking of the pulse wave icon PW when a blood pressure measurement result is displayed regardless of absence of an arrhythmia during blood pressure measurement. In addition, whether or not information reproducing the pulsation timing of a pulse wave is displayed only when an arrhythmia is present in this manner may be selected with an operation button of the operation unit 140.

When a past blood pressure calculation result is read out from the storage unit 130 to be checked, only a blood pressure measurement result in which an arrhythmia is detected by the arrhythmia detection unit 102 may be extracted.

In the above embodiment, when a blood pressure measurement result is displayed, the pulsation timing of a pulse wave, that is, pulse wave intervals are reproduced by blinking the pulse wave icon PW, but information to be reproduced is not limited to the pulse wave intervals. For example, information related to the amplitude of a pulse wave may be reproduced. Specifically, the magnitude of the amplitude may be reproduced by a difference in the type or brightness of the pulse wave icon PW, a display of a level gauge indicating the amplitude, or the like. In addition, also in the case of reproduction by sound, amplitude information of a pulse wave can be presented by the magnitude of a sound pressure level, the magnitude of the frequency of a sound, or the like.

In the above embodiment, the blood pressure measurement device 1 includes the main body portion 11 and the cuff portion 12 as separate members, but the present invention is also applicable to an integrated blood pressure monitor in which a main body portion is provided at a belt portion of a cuff, or a watch-type wearable blood pressure monitor worn at a wrist.

### REFERENCE NUMERALS LIST

1 Blood pressure measurement device
11 Main body portion
12 Cuff portion
13 Air tube
151 Image display portion
100 Control unit
110 Sensor unit
120 Cuff pressure control system
130 Storage unit
140 Operation unit
150 Image display unit
160 Sound output unit
T Measurement time
SBP Maximum blood pressure value
DBP Minimum blood pressure value
HB Pulse rate
LI Blood pressure value level indicator
S Attachment state icon
A Arrhythmia notification icon
PW Pulse wave icon

1-7. (canceled)

8. A blood pressure measurement device comprising:
a pulse wave acquisition unit configured to acquire a pulse wave of a human body;
a blood pressure value measurement unit configured to calculate a blood pressure value based on the pulse wave;
a pulse wave reproduction information generation unit configured to generate pulse wave reproduction information, the pulse wave reproduction information being dynamic information reproducing pulse wave intervals of the pulse wave at a time of calculation of the blood pressure value;
a storage unit configured to store the blood pressure value and the pulse wave reproduction information;
a display unit configured to display the blood pressure value; and
an output unit configured to output the pulse wave reproduction information,
wherein the output unit is configured to output the pulse wave reproduction information at the time of calculation of the blood pressure value when the blood pressure value stored in the storage unit is displayed at the display unit.

9. The blood pressure measurement device according to claim 8, further comprising an arrhythmia detection unit configured to detect an arrhythmia when the arrhythmia is present in the pulse wave acquired, wherein
the output unit outputs the pulse wave reproduction information only when the blood pressure value calculated based on the pulse wave at a time of detection of the arrhythmia is displayed at the display unit.

10. The blood pressure measurement device according to claim 9, wherein the display unit and/or the output unit output/outputs information indicating presence of the arrhythmia when the blood pressure value calculated based on the pulse wave at the time of detection of the arrhythmia is displayed at the display unit.

11. The blood pressure measurement device according to claim 8, wherein
the display unit also serves as the output unit, and
the pulse wave reproduction information is output at least as a moving image.

12. The blood pressure measurement device according to claim 11, wherein
the output unit includes a vibrating member, and
the pulse wave reproduction information is output as blinking of an icon in synchronization with peak positions of an amplitude of the pulse wave at the display unit and is output as vibrations and stops of the blood pressure measurement device in synchronization with the peak positions of the amplitude of the pulse wave at the vibrating member.
